(19) United States
(12) Patent Application Publication
(10) Pub. No.: US 2021/0370295 A1
(43) Pub. Date: Dec. 2, 2021

(54) MICROFLUIDIC DEVICE AND BIOLOGICAL DETECTION METHOD USING THE MICROFLUIDIC DEVICE

(71) Applicant: Visionatics Inc., TAINAN CITY (TW)

(72) Inventors: Chih-Wei FANG, Tainan City (TW); Jin-Yi WU, Tainan City (TW); Te-Hsun WANG, Tainan City (TW)

(21) Appl. No.: 17/333,037

(22) Filed: May 28, 2021

(30) Foreign Application Priority Data
May 29, 2020 (TW) 109118173

(51) Int. Cl. B01L 3/00; G01N 33/543; C12Q 1/6813

(52) U.S. Cl. CPC B01L 3/502715; G01N 33/54366; C12Q 1/6813; B01L 2300/0864; B01L 2200/16; B01L 2300/087; B01L 2200/10

(57) ABSTRACT

A microfluidic device and a biological detection method using the microfluidic device are provided. The microfluidic device includes a sample mixing area, an optical indicator mixing area and an observation area. The sample mixing area stores barcode beads and configured to receive a test sample to mix the barcode beads with the test sample, in which the barcode beads correspond to different detecting targets. The optical indicator mixing area is configured to receive an optical indicator and the barcode beads to mix the barcode beads with the optical indicator. The observation area is configured to receive the barcode beads from the optical indicator mixing area. In the biological detection method, at first, images of the barcode beads are captured. Thereafter, images of reacted barcode beads are determined, and values of barcode patterns thereof are recognized to determine reacting targets of the test sample.

### RELATED APPLICATION

This application claims priority to Taiwan Application Serial Number 109118173, filed May 29, 2020, which is herein incorporated by reference in its entirety.

### BACKGROUND

### Field of Invention

The embodiments of the present invention relate to a microfluidic device and a biological detection method using the microfluidic device.

### Description of Related Art

In recent years, because of the vigorous development of biological detection technology, microfluidic detection chips are widely used for various biological detections. Taking Immunoassay or Nucleic acid hybridization as an example, the microfluidic detection chip stores plural solid-phase carriers, and plural biological identification elements are immobilized on the solid-phase carriers. The biological identification elements include capture antibodies or capture probes. A test sample (i.e., target antigen or nucleic acid) is mixed with the solid-phase carriers in the microfluidic test chip, such that the target antigen or the nucleic acid is bound on the biological identification elements on the solid-phase carriers. Then, an optical indicator is added into the microfluidic test chip, such that the reaction of the test sample can be observed on the microfluidic test chip (including color change under visible light, fluorescence reaction and luminescence reaction).

However, when a conventional microfluidic detection chip is used for a biological detection, only one type of biological identification element can be used at one time during the biological detection is performed. If various biological identification elements are desired to be used at one time for the biological detection, the conventional microfluidic detection chip may be designed to have a great area, and fabrication cost of the conventional microfluidic detection chip is increased accordingly.

### SUMMARY

An object of the present invention is to provide a microfluidic device and a biological detection method using the microfluidic device to solve the aforementioned problem.

In accordance with some embodiments, the microfluidic device includes a sample mixing area, an optical indicator mixing area and an observation area. The sample mixing area stores a plurality of barcode beads, in which the sample mixing area is configured to receive a test sample to mix the barcode beads with the test sample, and the barcode beads correspond to different detecting targets. The optical indicator mixing area is configured to receive an optical indicator and the barcode beads to mix the barcode beads with the optical indicator. The observation area is configured to receive the barcode beads from the optical indicator mixing area for analysis and observation on the barcode beads performed by a user.

In some embodiments, the microfluidic device further includes a reaction reagent mixing area disposed between the sample mixing area and the optical indicator mixing area. The reaction reagent mixing area is configured to receive a reaction reagent and to receive the barcode beads from the sample mixing area to mix the barcode beads with the reaction reagent.

In some embodiments, the barcode beads include a plurality of biological recognition elements, and the biological recognition elements correspond to the detecting targets.

In some embodiments, the microfluidic device includes an electrical connection circuit configured to receive electric power provided by an external circuit.

In accordance with some embodiments, the microfluidic device includes a sample mixing area, a first reaction reagent mixing area, a first optical indicator mixing area and an observation area. The sample mixing area stores a plurality of barcode beads. The sample mixing area is configured to receive a test sample to mix the barcode beads with the test sample, and the barcode beads correspond to different detecting targets. The first reaction reagent mixing area is configured to receive a first reaction reagent and to receive a plurality of first barcode beads among the barcode beads from the sample mixing area to mix the first barcode beads with the first reaction reagent. The first optical indicator mixing area is configured to receive a first optical indicator and to receive the first barcode beads from the first reaction reagent mixing area to mix the first barcode beads with the first optical indicator. The observation area is configured to receive the first barcode beads from the first optical indicator mixing area for analysis and observation on the first barcode beads performed by a user.

In some embodiments, the microfluidic device further includes a second reaction reagent mixing area and a second optical indicator mixing area. The second reaction reagent mixing area is configured to receive a second reaction reagent and to receive a plurality of second barcode beads among the barcode beads from the sample mixing area to mix the second barcode beads with the second reaction reagent. The observation area is further configured to receive the second barcode beads from the second optical indicator mixing area for analysis and observation on the second barcode beads performed by the user.

In some embodiments, the barcode beads include a plurality of biological recognition elements, and the biological recognition elements correspond to the detecting targets.

In some embodiments, the microfluidic device further includes an electrical connection circuit configured to receive electric power provided by an external circuit.

In accordance with some embodiments, the biological detection method using the microfluidic device includes: providing the microfluidic device; capturing a plurality of barcode bead images of the barcode beads in the observation area; determining at least one reacted barcode bead image in accordance with the barcode bead images; recognizing a value of at least one barcode pattern of the at least one reacted barcode bead image; and determining at least one reacting target to which the test sample corresponds in accordance with the value of the at least one barcode pattern, wherein the at least one reacting target is at least one of the detecting targets.

In some embodiments, the barcode beads include a plurality of biological recognition elements, and the biological recognition elements correspond to the detecting targets.

### BRIEF DESCRIPTION OF THE DRAWINGS

Aspects of the present disclosure are better understood from the following detailed description in conjunction with the accompanying figures. It is noted that in accordance with the standard practice in the industry, various features are not drawn to scale. In fact, the dimensions of the various features can be arbitrarily increased or reduced for clarity of discussion.

FIG. 1 is a schematic diagram showing a microfluidic device in accordance with an embodiment of the present invention.

FIGS. 2A-2D are schematic diagrams showing the detection process performed by the microfluidic device.

FIG. 3 is a schematic diagram showing a microfluidic device in accordance with an embodiment of the present invention.

FIG. 4 is a schematic diagram showing a microfluidic device in accordance with an embodiment of the present invention.

FIG. 5 is a flow chart showing a biological detection method corresponding to the microfluidic device.

FIG. 6A is a schematic diagram showing a top view of a magnetic bead in accordance with an embodiment of the present invention.

FIG. 6B is a schematic diagram showing a side view of the magnetic bead in accordance with an embodiment of the present invention.

FIG. 7A and FIG. 7B are schematic diagrams showing magnetic beads having different values in accordance with embodiments of the present invention.

### DETAILED DESCRIPTION

The embodiments of the present disclosure are discussed in detail below. However, it will be appreciated that the embodiments provide many applicable concepts that can be implemented in various specific contents. The embodiments discussed and disclosed are for illustrative purposes only and are not intended to limit the scope of the present disclosure. In addition, the terms “first”, “second”, and the like, as used herein, are not intended to mean a sequence or order, and are merely used to distinguish elements or operations described in the same technical terms.

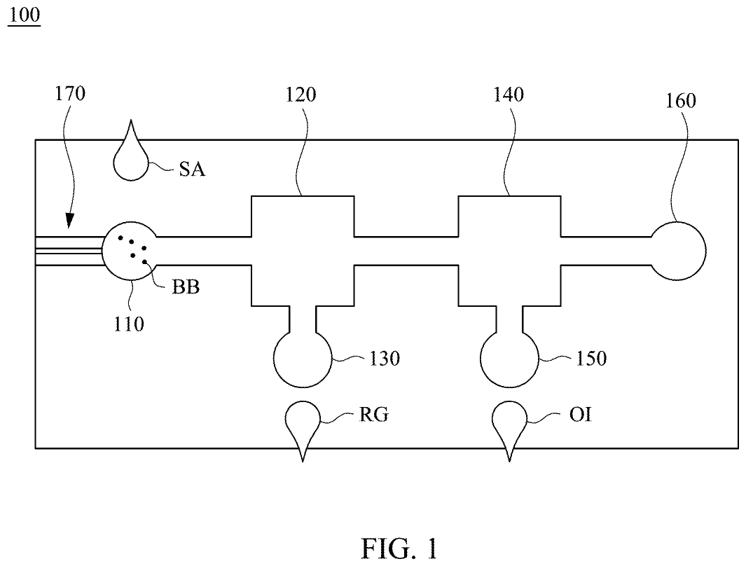

Referring to FIG. 1, FIG. 1 is a schematic diagram showing a microfluidic device 100 in accordance with an embodiment of the present invention. In this embodiment, the microfluidic device 100 is a microfluidic detection chip using an enzyme-linked immunosorbent assay (ELISA) or nucleic acid hybridization to perform a biological detection for a desired detecting target. The microfluidic device 100 includes a sample mixing area 110, a reaction reagent mixing area 120, a reagent storing area 130, an optical indicator mixing area 140, an optical indicator storing area 150 and an observation area 160. The sample mixing area 110 stores droplets, and the droplets includes a plurality of barcode beads BB. In this embodiment, the barcode beads have micro barcodes, for example those described in U.S. Pat. No. 8,590,794, having patterns representing numerical values. The sample mixing area 110 stores the barcode beads BB representing different numerical values, and the barcode beads BB representing different numerical values correspond to different detecting targets.

For example, the microfluidic device 100 includes three types of barcode beads BB which represent numerical values 1, 2 and 3, and a number of each of the three types of barcode beads BB can be determined in accordance with actual demands. For example, the number of each of the three types of barcode beads BB is 100. The barcode beads BB representing numerical value “1” correspond to a first allergen, and capture antibodies for the first allergen are immobilized thereon. The barcode beads BB representing numerical value “2” correspond to a second allergen, and capture antibodies for the second allergen are immobilized thereon. The barcode beads BB representing numerical value “3” correspond to a third allergen, and capture antibodies for the third allergen are immobilized thereon.

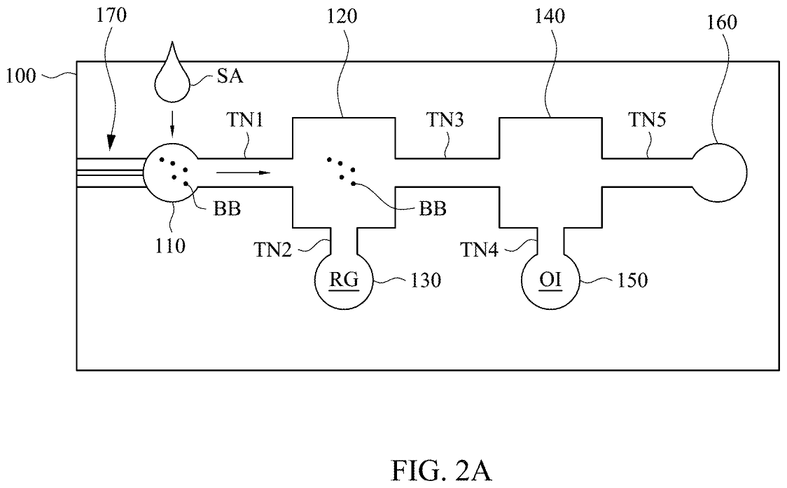

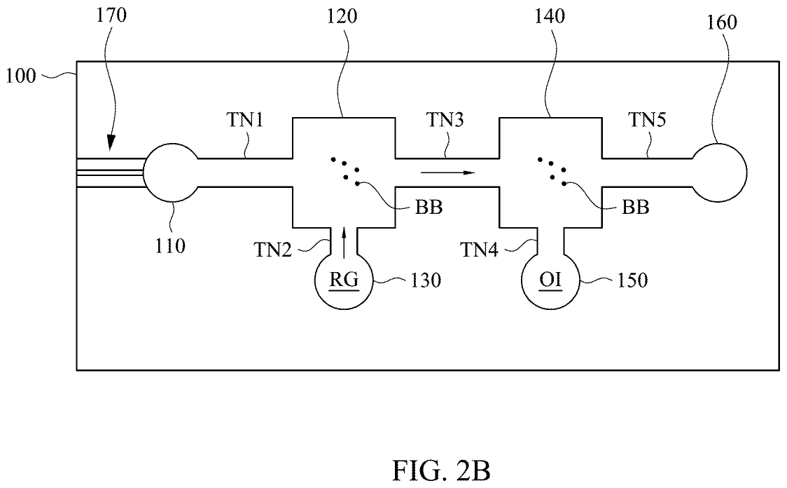

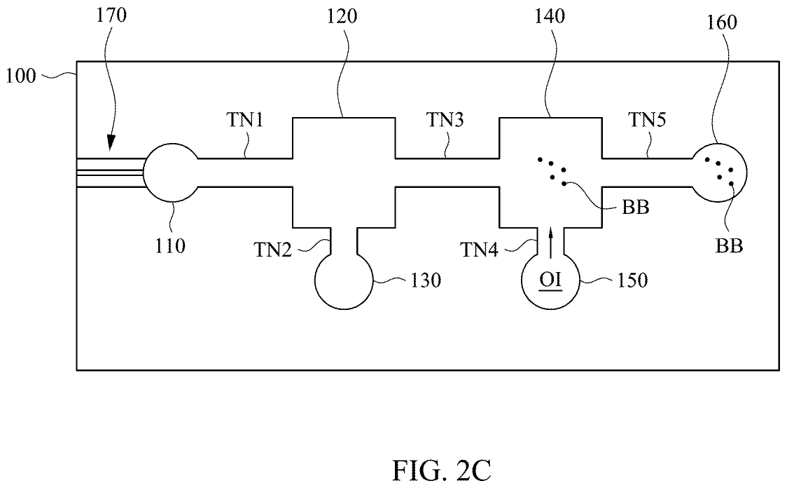

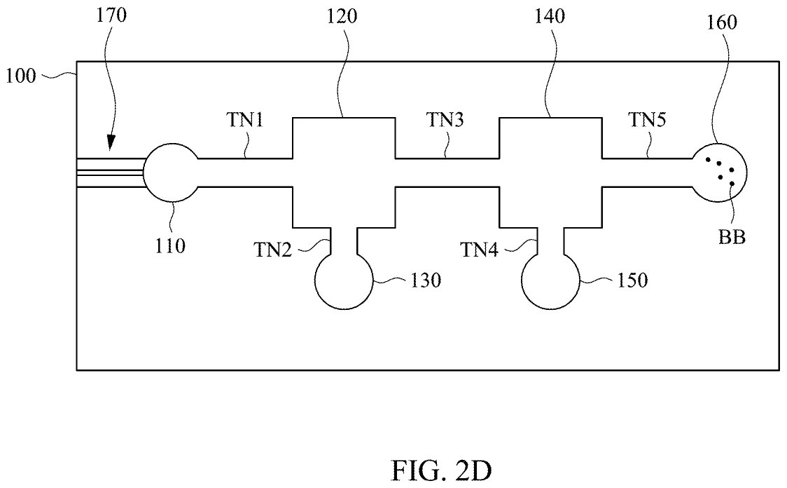

The sample mixing area 110 not only stores the three types of barcode beads BB, but also receives a test sample SA, for example serum. After the test sample SA is added into the sample mixing area 110, the droplets having the barcode beads BB are mixed with the test sample SA. Then, the droplets mixed with the test sample SA carry the barcode beads BB to flow into the reaction reagent mixing area 120 via a tunnel TN1, as shown in FIG. 2A. In this embodiment, the microfluidic device 100 includes an electrical connection circuit 170 configured to receive electric power provided by an external circuit, thereby driving the droplets in the sample mixing area 110 to flow, but embodiments of the present invention are not limited thereto. In other embodiments of the present invention, other methods, such as siphon can be used to drive the droplets in the sample mixing area 110.

The reaction reagent mixing area 120 is configured to receive the droplets mixed with the test sample SA and a reagent RG to mix the droplets with the reagent RG. In this embodiment, the reagent RG is stored in the reagent storing area 130 and includes probes corresponding to the test sample SA. When the droplets mixed with the test sample SA flow into the reaction reagent mixing area 120, the reagent storing area 130 can provide the reagent RG to the reaction reagent mixing area 120 via a tunnel TN2 to allow the reaction between the test sample SA and the reaction reagent RG. For example, the barcode beads BB reacted with the test sample SA can capture the probes. In one embodiment, the tunnel TN2 has a valve to control the reaction reagent RG to flow into the reaction reagent mixing area 120, but embodiments of the present invention are not limited thereto. Then, the droplets mixed with the reaction reagent RG carry the barcode beads BB to flow into the optical indicator mixing area 140 via a tunnel TN3, as shown in FIG. 2B.

The optical indicator mixing area 140 is configured to receive the droplets mixed with the reaction reagent RG and an optical indicator OI (including, for example a fluorescent indicator or a color indicator) to mix the droplets with the optical indicator OI, thereby using the optical indicator OI to indicate the reacted barcode beads BB. For example, the optical indicator OI includes optical molecules. When the droplets mixed with the reaction reagent RG are mixed with the optical indicator OI, the probes captured by the barcode beads BB can capture the optical molecules to mark the reacted barcode beads BB. In this embodiment, the optical indicator OI is stored in the optical indicator storing area 150. When the droplets mixed with the reaction reagent RG flow into the optical indicator mixing area 140, the optical indicator storing area 150 can provide the optical indicator OI to the optical indicator mixing area 140 via a tunnel TN4. In one embodiment, the tunnel TN4 has a valve to control the optical indicator OI to flow into the optical indicator mixing area 140, but embodiments of the present invention are not limited thereto. Then, the droplets mixed with the optical indicator OI carry the barcode beads BB to flow into the observation area 160 via a tunnel TN5, as shown in FIG. 2C.

As shown in FIG. 2D, after all the barcode beads BB flow into the observation area 160, the user can observe the barcode beads BB in the observation area 160 and determine if there are reacted barcode beads BB in the observation area 160. For example, the reacted barcode beads BB have a color change caused by the effect of the optical indicator OI for recognition. The color change may include a color change of visible light, a color change of fluorescent light, or a color change of luminescence. Therefore, it can be understood that which allergen the test sample SA is reacted with through capturing an image of the observation area 160, determining if the barcode beads BB in the image have the color change, and recognizing the value of each of the barcode beads BB having the color change. Specifically, if the barcode beads BB representing a value 1 and a value 3 have the color change, it can be considered that the test sample SA is reacted with the allergens represented by the barcode beads BB representing the value “1” and the value “3”.

Based on the above descriptions, the microfluidic device 100 of the embodiments of the present invention includes plural barcode beads BB corresponds to various different detecting targets, and these barcode beads BB enables the microfluidic device 100 to be convenient for biological detections and analysis simultaneously performed on the different detecting targets. Therefore, the microfluidic device 100 has a simple structure of flow channels, and does not require a large area for the structure of flow channels.

In some embodiments of the present invention, the reaction reagent mixing area 120 and the reagent storing area 130 can be removed. For example, the reagent RG is stored in the sample mixing area 110 in advance, and thus the test sample SA can be immediately reacted with the reagent RG when the test sample SA is added into the sample mixing area 110. Therefore, it is not required to dispose the reaction reagent mixing area 120 and the reagent storing area 130 in the microfluidic device 100.

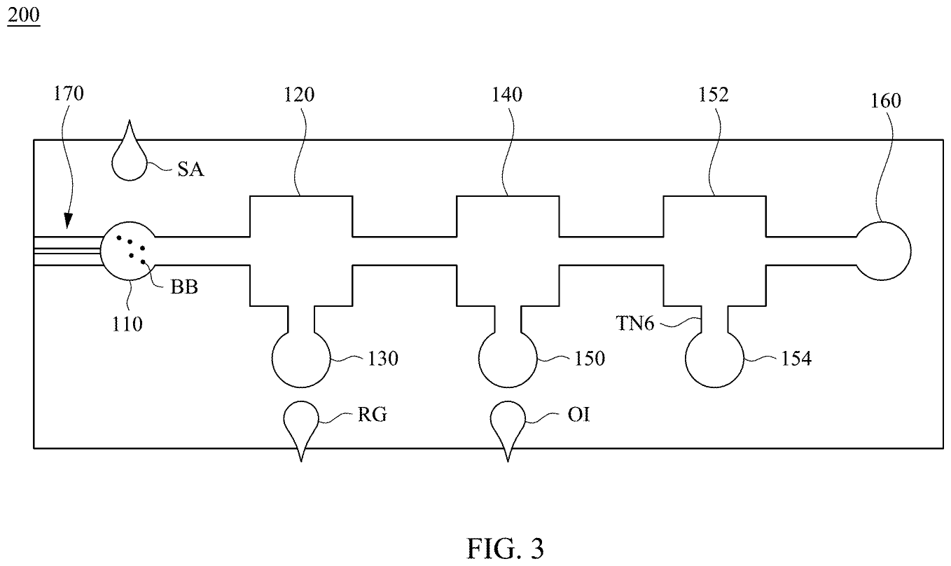

Referring to FIG. 3, FIG. 3 is a schematic diagram showing a microfluidic device 200 in accordance with an embodiment of the present invention. The microfluidic device 200 is similar to the microfluidic device 100, but the difference is in that the microfluidic device 200 further includes a cleansing area 152 and a washing buffer storing area 154. The cleansing area 152 is disposed between the optical indicator mixing area 140 and the observation area 160 to receive the droplets mixed with the optical indicator OI and a washing buffer (for example, water) and to mix the droplets with washing buffer, thereby removing (cleansing) the optical molecules not captured by the barcode beads BB though using the washing buffer. In this embodiment, the washing buffer is stored in the washing buffer storing area 154. When the droplets mixed with the optical indicator OI flows into the cleansing area 152, the washing buffer storing area 154 can provide the washing buffer to the cleansing area 152 via a tunnel TN6. In one embodiment, the tunnel TN6 has a valve to control the washing buffer to flow into the cleansing area 152, but embodiments of the present invention are not limited thereto. Thereafter, the cleansed droplets carry the barcode beads BB to flow into the observation area 160.

This embodiment uses the washing buffer to remove the optical molecules not captured by the barcode beads BB in the droplets, and thus the affection caused by the optical molecules not captured by the barcode beads BB can be avoid to reduce image background noise caused by the optical molecules not captured by the barcode beads BB. In one embodiment, an electromagnet (not shown) is disposed on the bottom of the cleansing area 152. When the washing buffer is used to remove the optical molecules not captured by the barcode beads BB, the electromagnet can be used to fix the barcode beads BB in the cleansing area 152. Therefore, when the waste liquid is generated after the remove (cleansing) of the optical molecules and flows back to the washing buffer storing area 154, the barcode beads BB remain in the washing buffer storing area 154.

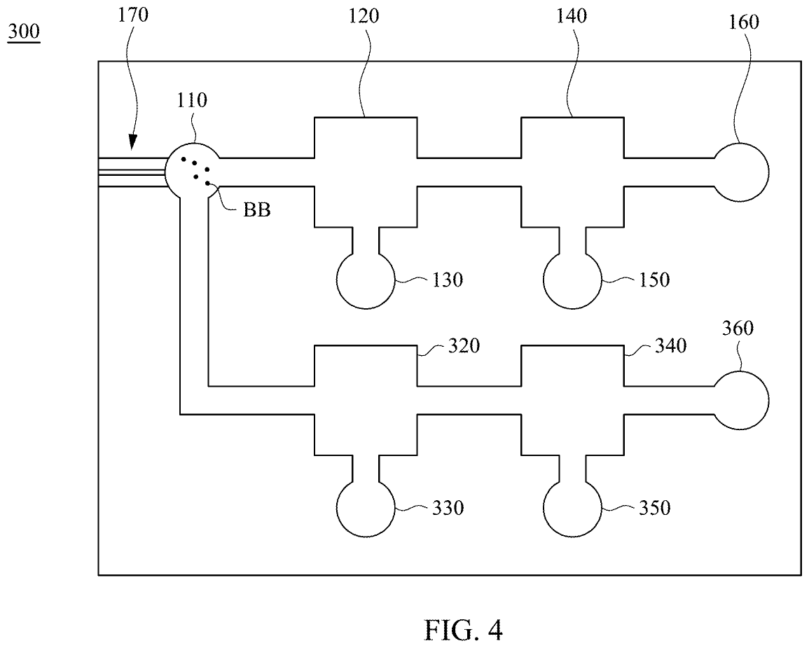

Referring to FIG. 4, FIG. 4 is a schematic diagram showing a microfluidic device 300 in accordance with an embodiment of the present invention. The microfluidic device 300 is similar to the microfluidic device 100, but the difference is in that the microfluidic device 300 further includes a reaction reagent mixing area 320, a reagent storing area 330, an optical indicator mixing area 340, an optical indicator storing area 350 and an observation area 360. The functions of the reaction reagent mixing area 320, the reagent storing area 330, the optical indicator mixing area 340, the optical indicator storing area 350 and the observation area 360 are similar to the functions of the reaction reagent mixing area 120, the reagent storing area 130, the optical indicator mixing area 140, the optical indicator storing area 150 and the observation area 160.

It is considered that different allergen detections may require different reaction reagents and/or optical indicators, the microfluidic device 300 of this embodiment provides another flow channel set for allergen detections. For example, after the droplets are mixed with the test sample SA in the sample mixing area 110, the droplets in the sample mixing area 110 are separated into two portions which individually flow into the reaction reagent mixing area 120 and the reaction reagent mixing area 320. The reaction reagent mixing area 320 is configured to receive a reagent and one of the portions of the droplets to mix the one of the portions of the droplets with the reagent. The optical indicator mixing area 340 is configured to receive the droplets provided from the reaction reagent mixing area 320 and to mix the droplets with the optical indicator. The observation area 360 is configured to provide an area for user's observation and analysis performed on the barcode beads BB in the droplets provided from the optical indicator mixing area 340.

In this embodiment, the reagent stored in the reagent storing area 330 is different from the reagent stored in the reagent storing area 130. In another embodiment of the present invention, the optical indicator stored in the optical indicator storing area 350 is different from the optical indicator stored in the optical indicator storing area 150. Therefore, the microfluidic device 300 can be adapted for a biological detection method using various different reagents.

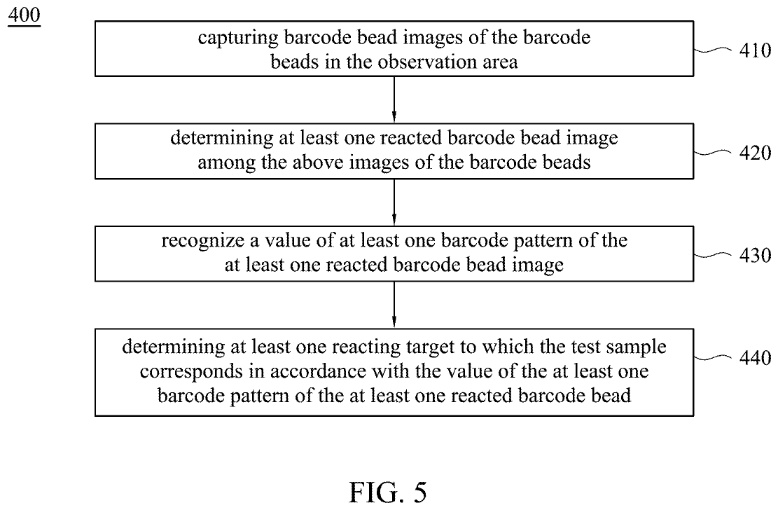

Referring to FIG. 5, FIG. 5 is a flow chart showing a biological detection method 400 corresponding to the microfluidic device 100. In the biological detection method 400, at first, step 410 is performed to capture images of the barcode beads BB in the observation area 160. For example, a camera can be used to capture an image of the observation area 160, and the observation area image includes all images of the barcode beads BB in the observation area 160. Then, step 420 is performed to determine at least one reacted barcode bead image (i.e., an image of a reacted barcode bead) among the above images of the barcode beads BB. For example, with respect to a target barcode bead image, a position of an area for fluorescent reading (for example, a pattern configured to emit fluorescent light) of the target barcode bead image is firstly checked. Then, a strength of optical signal of the target barcode bead image is determined in accordance with the gray level of the position of the area for fluorescent reading. If the strength of optical signal of the target barcode bead image is higher than a threshold value, it is determined that the barcode bead BB to which the target barcode bead image corresponds is reacted with an allergen.

Then, step 430 is performed to recognize a value of at least one barcode pattern of the at least one reacted barcode bead image. Because the micro barcode of U.S. Pat. No. 8,590,794 is adopted in some embodiments of the present invention, the method for recognizing the barcode pattern of the reacted barcode bead BB is also referred to U.S. Pat. No. 8,590,794. Details are not repeated here. However, the embodiments of the present invention are not limited thereto. In other embodiments of the present invention, other type of micro barcode can be used, and step 430 can be changed according to the change of the type of micro barcode.

Thereafter, step 440 is performed to determine at least one reacting target to which the test sample corresponds in accordance with the value of the at least one barcode pattern of the at least one reacted barcode bead BB. For example, in a case that plural reacted barcode bead BB are determined, and the values of the reacted barcode bead BB are recognized as “1” and “3”, it is considered that the test sample SA is reacted with the reacting targets (allergens) represented by the barcode beads BB representing the value “1” and the value “3”.

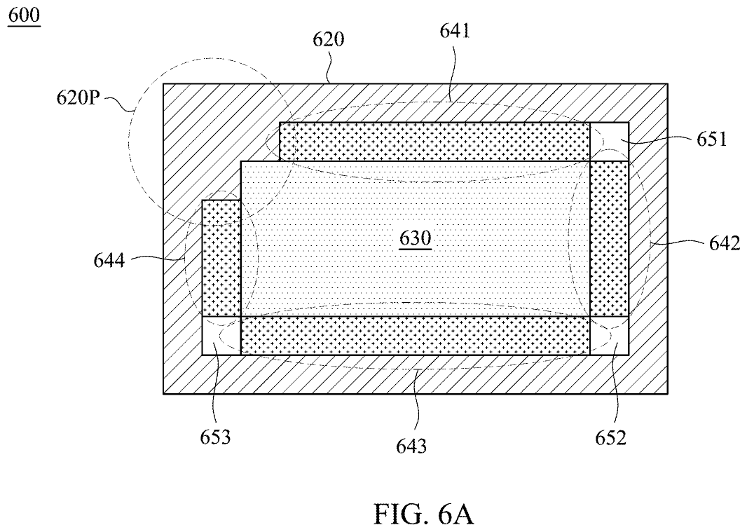

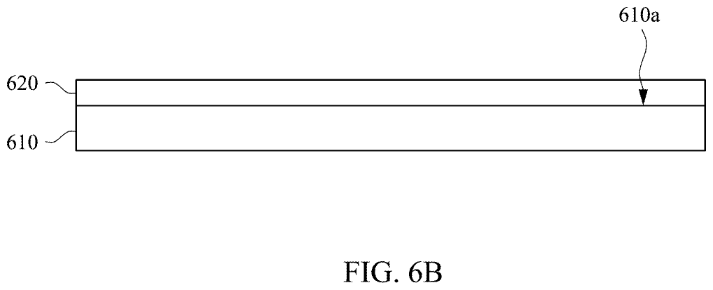

Referring to FIG. 6A and FIG. 6B, FIG. 6A is a schematic diagram showing a top view of a magnetic bead 600 in accordance with an embodiment of the present invention, and FIG. 6B is a schematic diagram showing a side view of the magnetic bead 600 in accordance with an embodiment of the present invention. In the above microfluidic device, the magnetic bead 600 can be used for replacement of the barcode bead BB. The magnetic bead 600 includes a substrate 610, a boundary pattern layer 620, a reaction pattern layer 630 and a data pattern layer including at least one sub data pattern layer. In this embodiment, the data pattern layer includes sub data pattern layers 641-644, but embodiments of the present invention are not limited thereto.

The boundary pattern layer 620 is disposed on a substrate surface 610a. In this embodiment, the boundary pattern layer 620 is disposed along the edges of the substrate 610, and a head portion and an end portion of the boundary pattern layer 620 are connected to each other to form a closed pattern, but embodiments of the present invention are not limited thereto. In other embodiments of the present invention, the head portion and the end portion of the boundary pattern layer 620 are not connected to each other. The boundary pattern layer 620 includes a positioning pattern 620P to help the positioning of the magnetic bead 600. The boundary pattern layer 620 is configured to provide counterweight of the magnetic bead 600 to achieve magnetic separation of magnetic bead 600. In this embodiment, the material of the boundary pattern layer 620 is Fe₃O₄, and the material of the substrate 610 is silicon, but embodiments of the present invention are not limited thereto. In some embodiments of the present invention, the boundary pattern layer 620 does not include magnetic material.

The reaction pattern layer 630 is disposed on the substrate surface 610a, and located in the area surrounded by the boundary pattern layer 620. In this embodiment, the reaction pattern layer 630 is disposed corresponding to a center of the substrate 610, but embodiments of the present invention are not limited thereto.

The reaction pattern layer 630 is configured to provide a result of reaction of a reaction reagent, and thus the material of the reaction pattern layer 630 is determined in accordance with contents of user's experiment. In this embodiment, the reaction pattern layer 630 is a fluorescent pattern layer, but embodiments of the present invention are not limited thereto. In other embodiments of the present invention, the reaction pattern layer 630 can be an acid-base indicator pattern layer. The material of the acid-base indicator pattern layer can be, for example phenol red. Specifically, the color of the phenol red is yellow at low pH, the color of the phenol red is red at high pH, and the color of the phenol red is orange between about pH 6.6 and about pH 8.0.

The data pattern layer is disposed on the substrate surface 610a and located between the boundary pattern layer 620 and the reaction pattern layer 630. In this embodiment, blank positioning points 651-653 are disposed between the sub data pattern layers 641-644 to separate the sub data pattern layers 641-644 from each other. In this embodiment, the material of the data pattern layer is Fe₃O₄, but embodiments of the present invention are not limited thereto. In some embodiment of the present invention, the data pattern layer does not include magnetic material.

As shown in FIG. 6A, in this embodiment, the substrate 610 is in a rectangular shape, and the reaction pattern layer 630 is in a rectangular shape, too. The positioning pattern 620P and the blank positioning points 651-653 correspond to the four vertexes of the substrate 610, respectively. For example, the positioning pattern 620P corresponds to a top-left vertex of the substrate 610, the blank positioning point 651 corresponds to a top-right vertex of the substrate 610, the blank positioning point 652 corresponds to a bottom-right vertex of the substrate 610, and the blank positioning point 653 corresponds to a bottom-left vertex of the substrate 610. Further, the sub data pattern layer 641 of the data pattern layer corresponds to long sides of the substrate 610 and the reaction pattern layer 630, the sub data pattern layer 642 of the data pattern layer corresponds to short sides of the substrate 610 and the reaction pattern layer 630, the sub data pattern layer 643 of the data pattern layer corresponds to long sides of the substrate 610 and the reaction pattern layer 630, and the sub data pattern layer 644 of the data pattern layer corresponds to short sides of the substrate 610 and the reaction pattern layer 630.

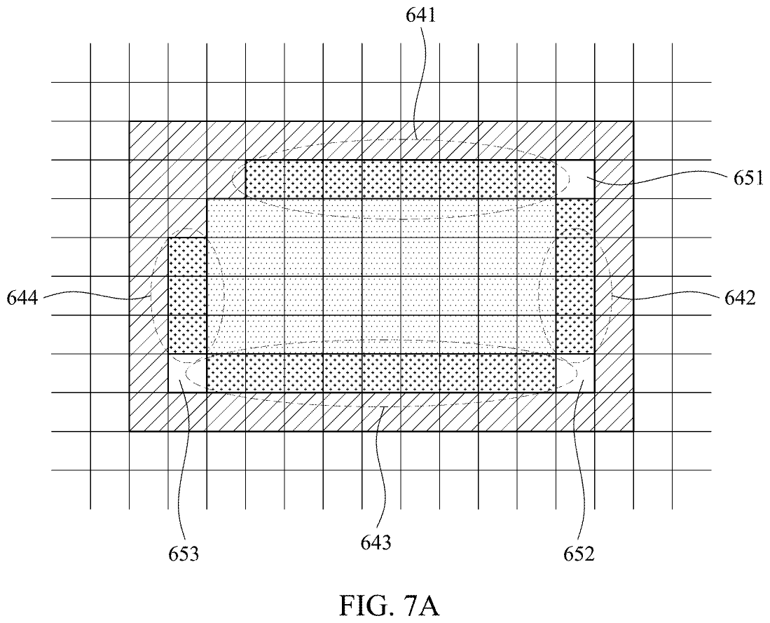

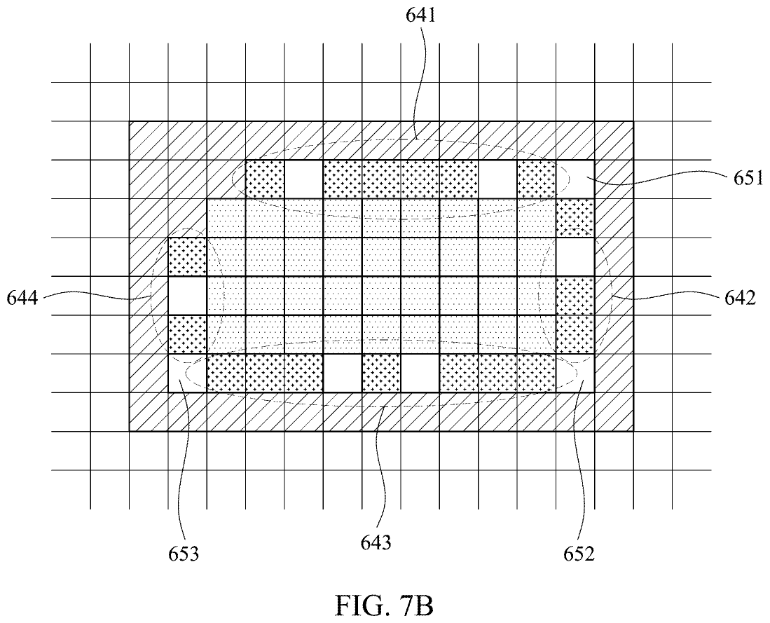

Referring to FIG. 7A and FIG. 7B, FIG. 7A and FIG. 7B are schematic diagrams showing magnetic beads 600 having different values in accordance with embodiments of the present invention. For convenience of illustration, grids are shown in FIG. 7A and FIG. 7B for explanation of the data types of the data pattern layer. In the embodiments of the present invention, the sub data pattern layer 641 includes eight blocks (hereinafter referred to as data blocks), and thus the sub data pattern layer 641 can store data of 8 bits. Similarly, the sub data pattern layer 642 includes four data blocks, and thus the sub data pattern layer 642 can store data of 4 bits. Similarly, the sub data pattern layer 643 includes nine data blocks, and thus the sub data pattern layer 643 can store data of 9 bits. Similarly, the sub data pattern layer 644 includes three data blocks, and thus the sub data pattern layer 644 can store data of 3 bits.

As shown in FIG. 7A, the eight data blocks of the sub data pattern layer 641 are all solid blocks, and thus a value of the data stored by the sub data pattern layer 641 can be considered to be 11111111. Similarly, the four data blocks of the sub data pattern layer 642 are all solid blocks, and thus a value of the data stored by the sub data pattern layer 642 can be considered to be 1111. Similarly, the nine data blocks of the sub data pattern layer 643 are all solid blocks, and thus a value of the data stored by the sub data pattern layer 643 can be considered to be 111111111. Similarly, the three data blocks of the sub data pattern layer 644 are all solid blocks, and thus a value of the data stored by the sub data pattern layer 644 can be considered to be 111. In this embodiment, a direction for data-reading is clockwise, and a start point of the data is the positioning pattern 620P. Therefore, a value of the data stored by the data pattern layer shown in FIG. 7A is 11111111-1111-111111111-111. In other embodiments of the present invention, the direction for data-reading and the start point of the data can be customized by the user. For example, in another embodiment of the present invention, a direction for data-reading is counterclockwise, and a start point of the data is the blank positioning point 652. Therefore, a value of the data stored by the data pattern layer shown in FIG. 7A is 1111-11111111-111-111111111.

As shown in FIG. 7B, the sub data pattern layer 641 has two data blocks which are blank blocks, and thus a value of the data stored by the sub data pattern layer 641 can be considered to be 10111101. Similarly, the sub data pattern layer 642 has one data block which is a blank block, and thus a value of the data stored by the sub data pattern layer 642 can be considered to be 1011. Similarly, the sub data pattern layer 643 has two data blocks which are blank blocks, and thus a value of the data stored by the sub data pattern layer 643 can be considered to be 111010111. Similarly, the sub data pattern layer 644 has one data block which is a blank block, and thus a value of the data stored by the sub data pattern layer 644 can be considered to be 101. In this embodiment, a direction for data-reading is clockwise, and a start point of the data is the positioning pattern 620P. Therefore, a value of the data stored by the data pattern layer shown in FIG. 7B is 10111101-1011-111010111-101. In other embodiments of the present invention, the direction for data-reading and the start point of the data can be customized by the user. For example, in another embodiment of the present invention, a direction for data-reading is counterclockwise, and a start point of the data is the blank positioning point 652. Therefore, a value of the data stored by the data pattern layer shown in FIG. 7B is 1101-10111101-101-111010111.

Based on the above descriptions, in the magnetic bead 600 of the embodiments of the present invention, the reaction pattern layer 630 is disposed in the center of the magnetic bead 600, and the data pattern layer (for example, the sub data pattern layers 641-644) are disposed between the boundary pattern layer 620 and the reaction pattern layer 630, and thus there is no other pattern included by the reaction pattern layer 630 to benefit the recognition of the magnetic bead 600. Further, with respect to the data pattern layer of the magnetic bead 600 of the embodiments of the present invention, the value of the data blocks can also be determined in accordance with the determination of existence or non-existence of the pattern material (for example, Fe₃O₄). In the above embodiments, the solid data block represents a value “1”, and the blank data block represents a value “0”, but embodiments of the present invention are not limited thereto. In other embodiments of the present invention, the solid data block represents a value “0”, and the blank data block represents a value “1”.

Further, FIG. 7A to FIG. 7B show that the magnetic bead 600 of the embodiment of present invention can store data of 24 bits, but embodiments of the present invention are not limited thereto. A size of the magnetic bead 600 (the data bits can be stored) can be adjusted (enlarged or shrank) in accordance with the demands of the user to provide different storage of data bits. For example, in one embodiment of the present invention, the sub data pattern layer 641 may be shrank to have four data blocks, the sub data pattern layer 642 may be shrank to have two data blocks, the sub data pattern layer 643 may be shrank to have five data blocks, and the sub data pattern layer 644 may be shrank to have five data blocks. Therefore, the adjusted magnetic bead 600 can provide data storage of 12 bits.

The features of several embodiments are outlined above, so those skilled in the art can better understand the aspects of the present disclosure. Those skilled in the art will appreciate that the present disclosure can be readily utilized as a basis for designing or modifying other processes and structures, thereby achieving the same objectives and/or achieving the same advantages as the embodiments described herein. Those skilled in the art should also understand that these equivalent constructions do not depart from the spirit and scope of the present disclosure, and they can make various changes, substitutions and alteration without departing from the spirit and scope of the present disclosure.

What is claimed is:

1. A microfluidic device, comprising:
a sample mixing area storing a plurality of barcode beads, wherein the sample mixing area is configured to receive a test sample to mix the barcode beads with the test sample, and the barcode beads correspond to different detecting targets;
an optical indicator mixing area configured to receive an optical indicator and the barcode beads to mix the barcode beads with the optical indicator; and
an observation area configured to receive the barcode beads from the optical indicator mixing area for analysis and observation on the barcode beads performed by a user.

2. The microfluidic device of claim 1, further comprising a reaction reagent mixing area disposed between the sample mixing area and the optical indicator mixing area, and configured to receive a reaction reagent and to receive the barcode beads from the sample mixing area to mix the barcode beads with the reaction reagent.

3. The microfluidic device of claim 1, wherein the barcode beads comprise a plurality of biological recognition elements, and the biological recognition elements correspond to the detecting targets.

4. The microfluidic device of claim 1, further comprising an electrical connection circuit configured to receive electric power provided by an external circuit.

5. A microfluidic device, comprising:
a sample mixing area storing a plurality of barcode beads, wherein the sample mixing area is configured to receive a test sample to mix the barcode beads with the test sample, and the barcode beads correspond to different detecting targets;
a first reaction reagent mixing area configured to receive a first reaction reagent and to receive a plurality of first barcode beads among the barcode beads from the sample mixing area to mix the first barcode beads with the first reaction reagent;
a first optical indicator mixing area configured to receive a first optical indicator and to receive the first barcode beads from the first reaction reagent mixing area to mix the first barcode beads with the first optical indicator; and
an observation area configured to receive the first barcode beads from the first optical indicator mixing area for analysis and observation on the first barcode beads performed by a user.

6. The microfluidic device of claim 5, further comprising:
a second reaction reagent mixing area configured to receive a second reaction reagent and to receive a plurality of second barcode beads among the barcode beads from the sample mixing area to mix the second barcode beads with the second reaction reagent; and
a second optical indicator mixing area configured to receive a second optical indicator and to receive the second barcode beads from the second reaction reagent mixing area to mix the second barcode beads with the second optical indicator; and
wherein the observation area is further configured to receive the second barcode beads from the second optical indicator mixing area for analysis and observation on the second barcode beads performed by the user.

7. The microfluidic device of claim 5, wherein the barcode beads comprise a plurality of biological recognition elements, and the biological recognition elements correspond to the detecting targets.

8. The microfluidic device of claim 5, further comprising an electrical connection circuit configured to receive electric power provided by an external circuit.

9. A biological detection method using a microfluidic device, comprising:
providing the microfluidic device comprising:
a sample mixing area storing a plurality of barcode beads, wherein the sample mixing area is configured to receive a test sample to mix the barcode beads with the test sample, and the barcode beads correspond to different detecting targets;
an optical indicator mixing area configured to receive an optical indicator and the barcode beads to mix the barcode beads with the optical indicator; and
an observation area configured to receive the barcode beads from the optical indicator mixing area;
capturing a plurality of barcode bead images of the barcode beads in the observation area;
determining at least one reacted barcode bead image in accordance with the barcode bead images;
recognizing a value of at least one barcode pattern of the at least one reacted barcode bead image; and
determining at least one reacting target to which the test sample corresponds in accordance with the value of the at least one barcode pattern, wherein the at least one reacting target is at least one of the detecting targets.

10. The biological detection method of claim 9, the barcode beads comprise a plurality of biological recognition elements, and the biological recognition elements correspond to the detecting targets.
